(19) United States
(12) Patent Application Publication
(10) Pub. No.: US 2009/0143770 A1
(43) Pub. Date: Jun. 4, 2009

(54) Medical luer fitting that promotes liquid mixing

(76) Inventors: Mark Ries Robinson, Albuquerque, NM (US); Alan Ross, Albuquerque, NM (US); Donald James Novkov, Encinitas, CA (US)

Correspondence Address: V. Gerald Grafe, Esq., P.O. Box 2689, Corrales, NM, 87048, US

(21) Appl. No.: 12/325,245

(22) Filed: Nov. 30, 2008

Related U.S. Application Data
(60) Provisional application No. 60/991,447, filed on Nov. 30, 2007.
(60) Provisional application No. 60/992,037, filed on Dec. 3, 2007.

(51) Int. Cl. A61M 39/10

(52) U.S. Cl. 604/533

(57) ABSTRACT

A medical luer connection improves liquid mixing thereby reducing areas of low or stagnant flow within the plenum chamber formed between the male and female luer connectors. Stagnation or low flow areas in the plenum can be reduced by imparting a non-axial flow component to the fluid flow as it passes through the plenum. Improvement of the cleaning effectiveness of the luer connection reduces the amount of flushing fluid needed to clean the connection, reduces the residual blood matter at a fixed volume of fluid relative to a standard luer connection, or combinations of the above.

### CROSS-REFERENCE TO RELATED APPLICATIONS

This application claims the benefit of U.S. Provisional Application No. 60/991,447, filed Nov. 30, 2007, and U.S. Provisional Application No. 60/992,037, filed Dec. 3, 2007, both of which are incorporated herein by reference.

### FIELD OF THE INVENTION

The present invention relates to medical devices and, in particular, to a medical luer connector that promotes liquid mixing and effective cleaning of the luer connection.

### BACKGROUND OF THE INVENTION

Luer connectors are used in a variety of medical applications to interconnect tubing used in, for example, intravenous (IV) devices. A typical luer connection comprises a male luer connector that is inserted into a female luer connector. The ends of the male and female luers are constructed to securely and reliably engage so that fluid can be passed between them without escaping or leaking from the connection.

FIG. 1 shows a standard medical luer connection comprising a standard female luer connector 10 and a standard male luer connector 20. The female luer 10 comprises a hollow cylindrical housing 12 with a forwardly-tapered inner wall 14 that accepts the conical proximal end 24 of a tubular male luer body 22 to provide a secure liquid seal. As used herein, “distal” is the rearward end of the male luer and “proximal” is the forward end that is inserted into the rear end of the female luer. The luers can be securely engaged by threads or other locking means (not shown). Both the female and male luers have axial hollow bores 15 and 25 for passage of a working fluid therein. The forward end of the standard male luer has a flattened end face 26 thereby forming with the inner wall 14 of the female luer a hollow chamber, or plenum, 16 within the female luer housing 12 that allows fluid transfer between the bores of the two luers.

The standard medical IV luer connection is specified in the International Standard ISO 594/1. This ISO standard provides nominal dimensions and tolerances for female and male luers designed to connect together and thereby form a reliable seal. Although luers having the standard geometry work well for the basic requirement of making a leak-free connection, the standard geometry does not perform well for flushing residual liquid from the connection. With the standard geometry, as well as with slight variations thereof, a recirculating flow pattern develops within the plenum when liquid flows in either the forward or reverse direction through the luer connection. Due to the slow mixing resulting from this recirculating flow, considerable flushing liquid (e.g., saline solution) must be passed through the connection in order to flush the working liquid (e.g., blood) from the plenum.

As noted above, the ISO standard defines nominal dimensions so that a leak free connection can be formed. However, the volume of the plenum chamber can vary depending upon the dimensions of the female and male luers. FIG. 2 shows a cross-section side-view schematic illustration of a standard medical luer connection having maximum female luer inner wall and minimum male luer outer wall dimensions. The volume of the plenum chamber is very small. Such a small plenum has little room for stagnant flow and, therefore, can be cleaned with a moderate amount of flushing liquid. FIG. 3 is a cross-section side-view schematic illustration of a standard medical luer connection having minimum female luer inner wall and maximum male luer outer wall dimensions. With these dimensions, the volume of the plenum chamber is significantly bigger. The increased plenum size creates opportunities for fluid stagnation and recirculating flow patterns, therefore requiring a large amount of flushing liquid to clean the plenum.

In most applications, luer connectors are used to join infusion tubing. In such cases, the flow is unidirectional and the fluid being infused is unlikely to clot or aggregate in the luer and, specifically, in the plenum chamber. However, the desire for automated blood analyte measurements has increased and blood access lines are now being used to obtain blood from the patient for analysis. There are many types of blood access systems but almost all involve the removal of blood from the body and the re-infusion of some or all of the blood back into the body. The blood removal process results in blood flowing through a luer connection. In both animal and human blood testing, the standard luer connection demonstrated significant aggregation or clotting in areas of lowest flow. In particular, clots formed in areas where the flow stream simply passed between the axially aligned hollow bores of the female and male luers with little flow occurred near the plenum chamber walls.

FIG. 4 shows an exemplary blood access system used for blood analyte measurements. In the process of transporting the blood from the patient to the measurement sensor there are two luer connections in this system. The first luer is between the catheter in the patient and an extension set. Extension sets are commonly used to facilitate catheter placement. A second luer is used to connect the extension set to the blood access circuit. Both luer connections are exposed to blood and require cleaning if aggregation is to be avoided over time.

As the blood analyte measurements are typically intermittent in nature, the blood access system may initiate a cleaning or infusion process to clean the system so as to prevent clotting or aggregation of blood product, including fibrin, platelets and red blood cells, in the luer connection. The cleaning process may utilize significant amounts of flushing liquid for effective cleaning. The patient can become volume overloaded when these cleaning fluids, typically saline, are infused into the patient.

Therefore, a need remains for a luer connection that can be cleaned effectively and efficiently following exposure of the connection to blood.

### SUMMARY OF THE INVENTION

The present invention is directed to a medical male luer, comprising means to reduce areas of low or stagnant flow within the plenum chamber formed between male and female luer connectors. In luer connections, the tubular male luer body comprises an axial hollow bore for flow of fluid therethrough. The proximal end of the male luer connects to a female luer having a corresponding axial hollow bore, thereby forming a plenum at the connection. Reduction of stagnation or low flow areas in the plenum can be reduced by imparting a non-axial flow component to the fluid flow as it passes through the plenum. This non-axial flow component can be imparted via a variety of embodiments disclosed herein which can improve the cleaning effectiveness of the luer connection. Improvement of the cleaning effectiveness reduces the amount of flushing liquid needed to clean the connection, reduces the residual blood matter at a fixed volume of fluid relative to a standard luer connection, or combinations of the above.

The invention relates to a male luer that imparts a non-axial component to the fluid flow so as to more effectively clean the plenum chamber formed between the male and female luers. The male luer can impart a radial, rotational, and/or mixing component to the fluid flow so as to more effectively clean the plenum chamber. Finally, a male luer of the present invention can impart different flow paths depending upon the rate of flow within the luer connection, so as to more effectively clean the plenum chamber when the flow rate is varied.

### BRIEF DESCRIPTION OF THE DRAWINGS

The accompanying drawings, which are incorporated in and form part of the specification, illustrate the present invention and, together with the description, describe the invention. In the drawings, like elements are referred to by like numbers.

FIG. 1 is a cross-section side-view schematic illustration of a standard medical luer connection.

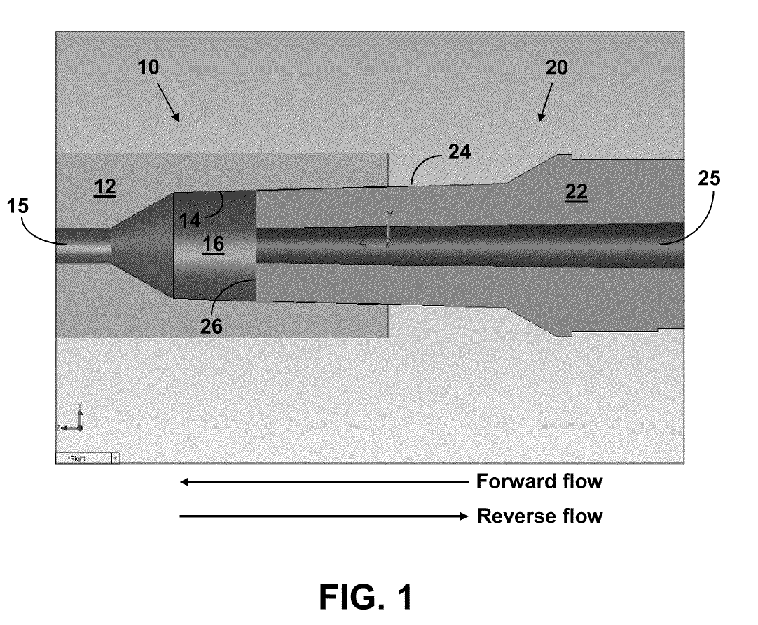

FIG. 2 is a cross-section side-view schematic illustration of a standard medical luer connection having maximum female luer inner wall and minimum male luer outer wall dimensions.

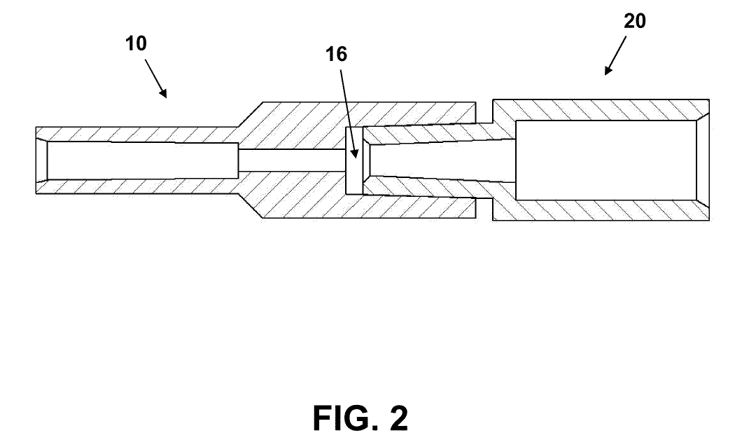

FIG. 3 is a cross-section side-view schematic illustration of a standard medical luer connection having minimum female luer inner wall and maximum male luer outer wall dimensions.

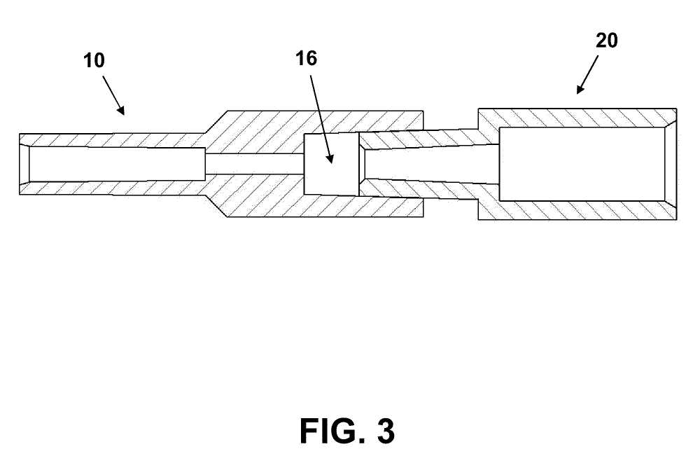

FIG. 4 is a schematic illustration of a blood access system for measurement of blood analytes.

FIG. 5 is a schematic illustration of a funnel luer connection.

FIG. 6 is a velocity contour plot at center cross section of a funnel luer connection with a 10 ml/min forward flow of water. Regions near the luer axis have a high flow velocity. Regions near the plenum wall have low-velocity recirculation zones.

FIG. 7 is a 3D flow trajectory plot of 30 streamlines for a funnel luer connection with a 10 ml/min forward flow of water.

FIG. 8 is a velocity contour plot at center cross section of a funnel luer connection with a 10 ml/min reverse flow of water. Regions near the luer axis have a high flow velocity. Regions near the plenum wall have low-velocity recirculation zones.

FIG. 9 is a 3D flow trajectory plot of 30 streamlines for a funnel luer connection with a 10 ml/min reverse flow of water.

FIG. 10 is a perspective view illustration of a tubular insert male luer.

FIG. 11 is a perspective view illustration of a mixing male luer.

FIG. 12 is a cross-section top view schematic illustration, in the x-z plane, of a mixing luer connection comprising a mixing male luer and a standard female luer.

FIG. 13 is a cross-section side view schematic illustration, in the y-z plane, of a mixing luer connection comprising a mixing male luer and a standard female luer.

FIG. 14 is a velocity contour plot at the center cross section of a mixing luer connection in the x-z plane with a 10 ml/min forward flow of water.

FIG. 15 is a velocity contour plot at the center cross section of a mixing luer connector in the y-z plane with a 10 ml/min forward flow of water.

FIG. 16 is a 3D flow trajectory plot of a mixing luer connection showing 30 streamlines with a 10 ml/min forward flow of water.

FIG. 17 is a velocity contour plot at center cross section of a mixing luer connection in the x-z plane with a 10 ml/min reverse flow of water.

FIG. 18 is a velocity contour plot at center cross section of a mixing luer connection in the y-z plane with a 10 ml/min reverse flow of water.

FIG. 19 is a 3D flow trajectory plot of a mixing luer connection showing 30 streamlines with a 10 ml/min reverse flow of water.

FIG. 20 is a cross-sectional side-view schematic illustration of a vortex luer connection.

FIG. 21 is a perspective end-view illustration of a vortex male luer.

FIG. 22 is a perspective side-view illustration of a vortex male luer.

FIG. 23 is a 3D flow trajectory plot of a vortex luer connection showing 30 streamlines with a 10 ml/min forward flow of water.

FIG. 24 is a cross-sectional side-view schematic illustration of a scoop luer connection.

FIG. 25 is a perspective end-view illustration of a scoop male luer.

FIG. 26 is a perspective side-view illustration of a scoop male luer.

FIG. 27 is a 3D flow trajectory plot of a scoop luer connection showing 30 streamlines with a 10 ml/min forward flow of water.

FIG. 28 is a bar graph of optical absorbance at 415 nm of blood residual cellular matter for three types of luer connections.

### DETAILED DESCRIPTION OF THE INVENTION

The present invention is directed toward a medical luer connector that achieves improved flushing performance by forcing the fluid flow at the luer connection into as much of the open zone of the plenum as possible, thus scouring the plenum, while substantially avoiding recirculation or stagnant flow.

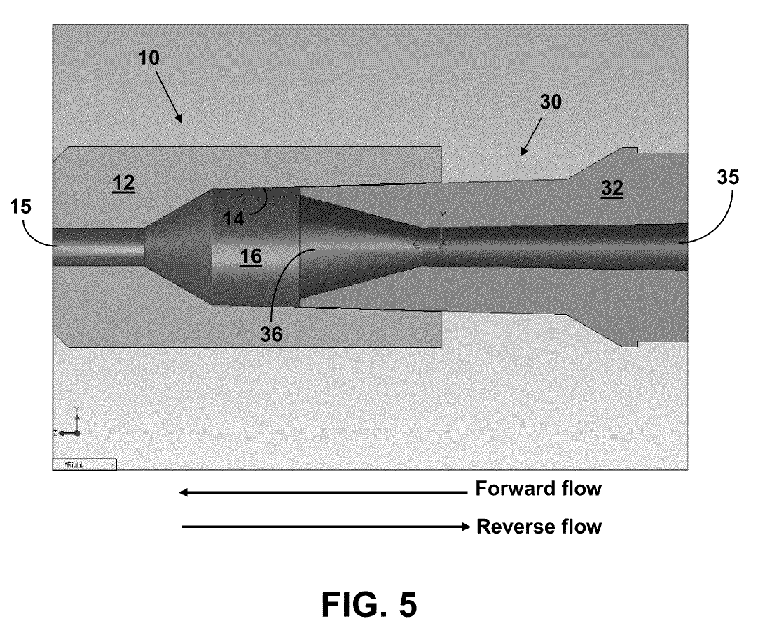

To enhance mixing within the plenum, the “funnel luer” connector was developed. FIG. 5 shows a funnel luer connection. The connection comprises a standard female luer 10 and a funnel male luer 30. The funnel male luer 30 has a similar geometry to the standard male luer 20, but instead of the flow passage being a straight hole passing all the way through the luer, the proximal end of the flow passage bore 35 is tapered outwardly to form a funnel 36. The effect of the funnel end of the male luer on fluid flow is different in the forward and reverse directions. In the forward direction (i.e., male to female luer direction), the funnel 36 provides for an expansion of the liquid streamlines, making use of the boundary layer attachment phenomenon to distort the streamlines into a larger zone of the plenum 16. In the reverse direction (i.e., female to male luer direction), the male luer end acts exactly as a funnel; that is, it allows liquid from a larger zone of the plenum 16 to flow directly into a converging flow stream that enters the flow passage bore 35 of the male luer at the apex of the funnel 36. In this manner, recirculation is reduced in both the forward and reverse directions. The funnel luer connection performs better than does the standard luer connection with respect to flushing liquid.

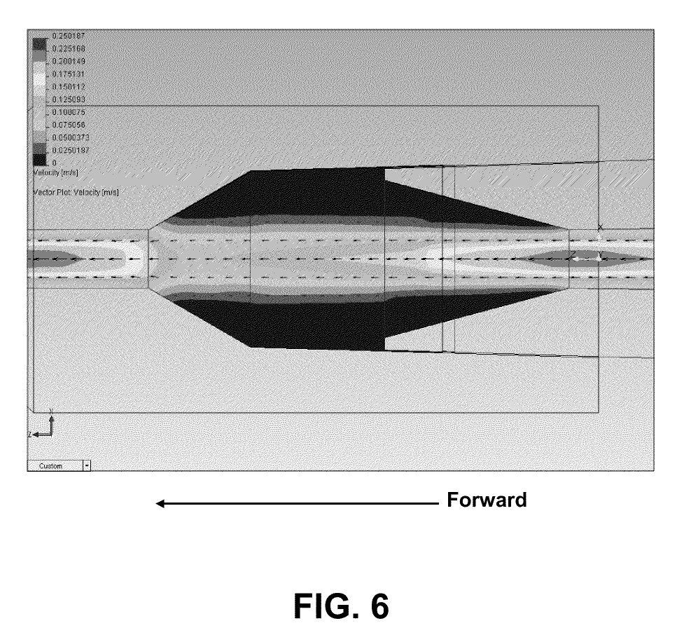

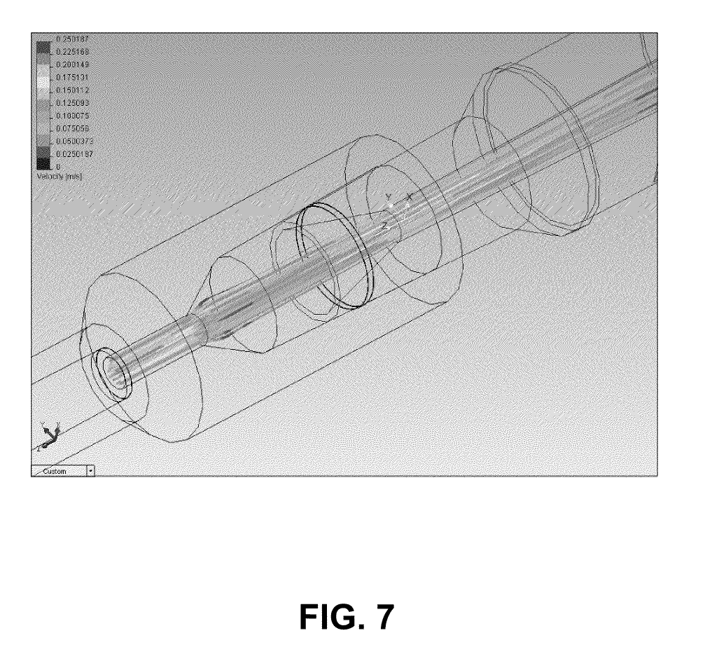

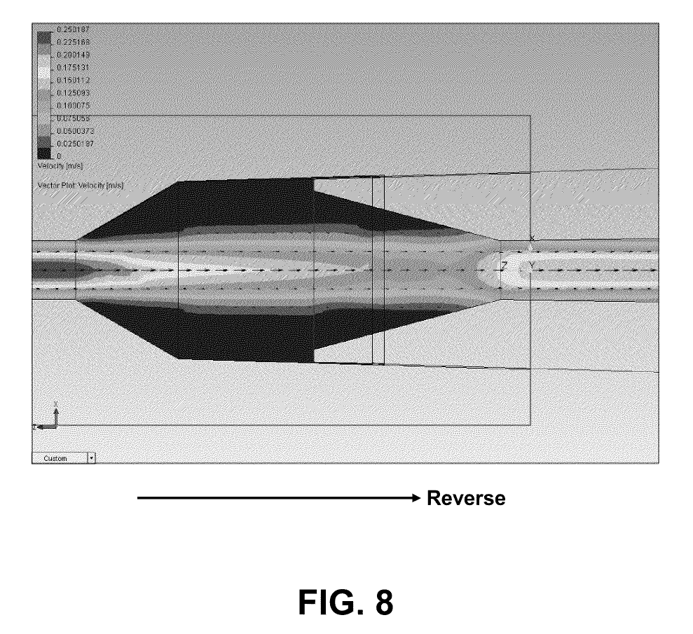

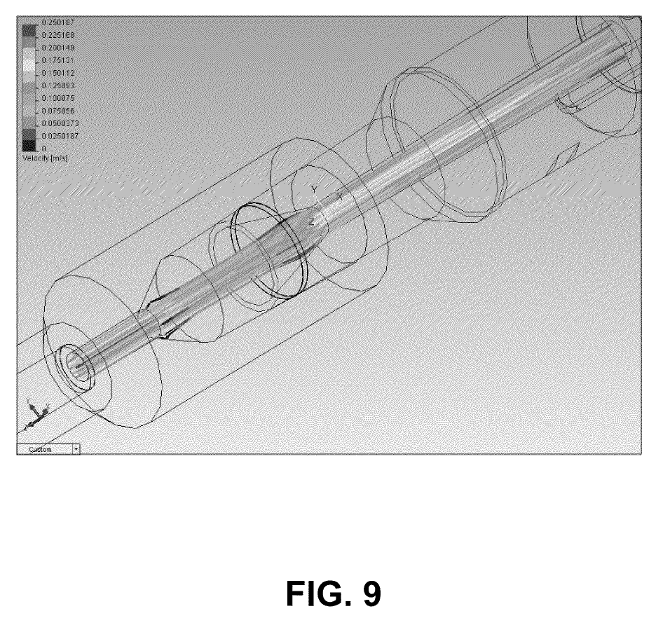

FIGS. 6-9 show analyses of the fluid flow in a funnel luer connection obtained using a 3D computational fluid dynamics (CFD) model. FIG. 6 shows a velocity contour plot at center cross section of a funnel luer connection with a 10 ml/min forward flow of water. As can be seen in the gray scale image, the center of the axial flow stream represents the area of greatest flow while the walls of the plenum chamber experience much less flow. These areas of little or no flow are problematic, since aggregation occurs in these low-flow areas. FIG. 7 shows a 3D flow trajectory plot of 30 streamlines with a 10 ml/min forward flow of water. The streamlines are predominantly in the axial direction with fluid passing directly between the axially aligned hollow bores of the male and female luers. FIG. 8 shows a velocity contour plot at center cross section of funnel luer connection with a 10 ml/min reverse flow of water. As can be seen in the gray scale image, the center of the axial flow stream represents the area of greatest flow while the walls of the plenum chamber experience little or no flow. FIG. 9 shows a 3D flow trajectory plot of 30 streamlines with a 10 ml/min reverse flow of water. Although better than a standard luer, the funnel luer connection is not an optimal solution due to the areas of low flow near the walls of the plenum. If the flow in the plenum chamber were more uniform, the areas of low flow could be reduced and the overall cleaning effectiveness of the connection could be improved.

Various other male luer geometries have been tested in order to enhance flushing performance. An “angled tip” luer is similar to the standard luer geometry except the male luer has a flow path that changes the flow angle such that some degree of radial outward flow is induced.

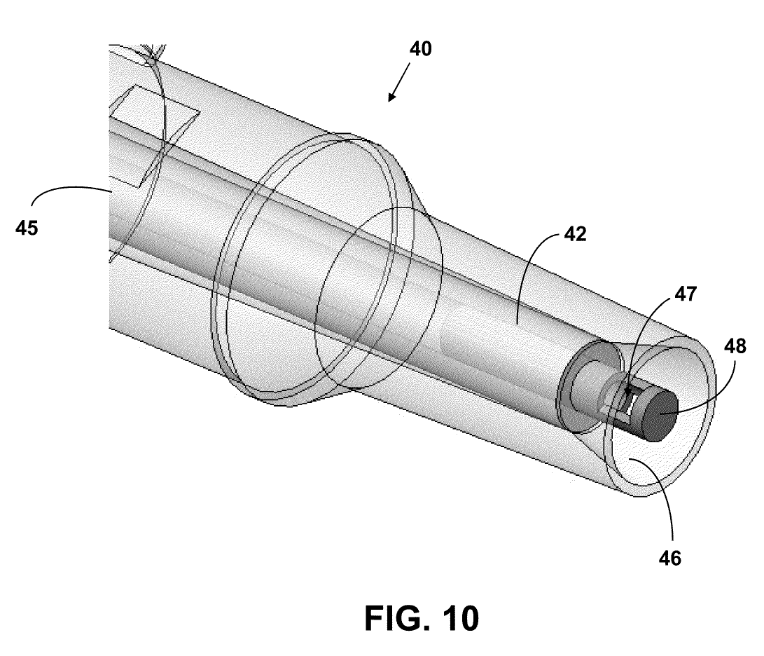

FIG. 10 shows a “tubular insert” male luer 40 that changes the fluid flow angle. The tubular insert male luer 40 comprises a closed-end, thin-walled hollow tube 42 inserted into the hollow bore 45 of a funnel male luer. The tubular insert can be a metal tube. The tube 42 has radial side ports, or orifices, 47 cut into its proximal end and an end cap 48 to close the end of the insert tube. This closed-end tube results in flow which is directed radially outward into the funnel zone 46 at the proximal end of the funnel male luer. The tubular insert luer demonstrates improved flushing performance. However, zones of low flow still exist, since there are only two exit ports for redirection of the fluid flow. In testing, the metal insert luer exhibited some degree of gravitational sensitivity. Specifically, if one of these low flow zones was assisted by gravity, aggregation could occur when the luer was flushed with low cleaning volumes.

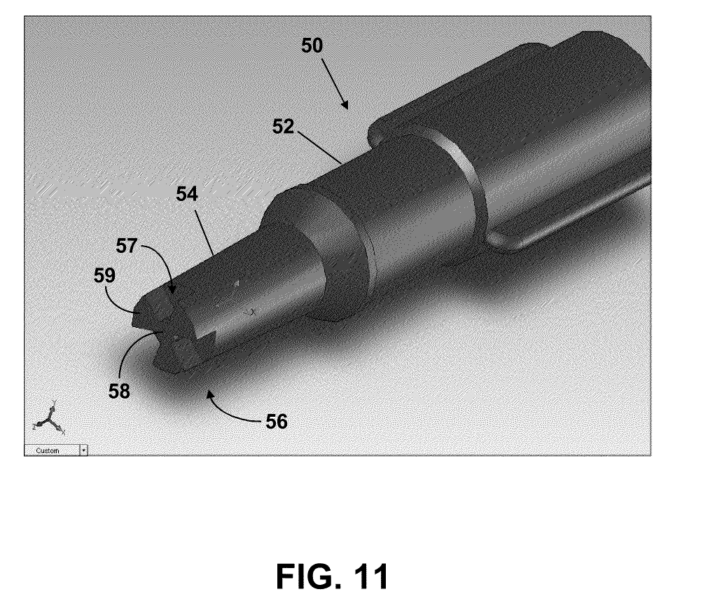

FIG. 11 shows a perspective view illustration of the proximal end of a “mixing” male luer 50 that has improved flushing performance. The mixing male luer 50 comprises an elongated tubular male luer body 52 comprising an axial hollow bore 55 for the flow of medical fluids therethrough. The distal end of the male luer can be connected to medical tubing for delivery of the medical fluid to the hollow bore of the luer. The male luer 50 has a conical proximal end 54 that can connect to a standard female luer or other type of female luer connector. As with the standard luer connection, the plenum within the female luer housing allows fluid transfer between the bores of the connected luers.

The proximal end of the mixing male luer 50 comprises a dispersing nozzle 56 wherein the nozzle receives fluid flowing forward in the axial hollow bore 55 and redirects the fluid radially outward through one or more slots or orifices 57 in the sidewall of the tubular male luer body 52 toward the inner walls of the plenum. The dispersing nozzle 56 can have an axial closed end cap 58, the inner surface of which redirects the axial bore flow radially outward. Alternatively, the flow can be reversed so that radially inward flowing fluid from the plenum can be redirected in the nozzle to flow axially through the bore toward the distal end of the male luer. The orifices can be formed from axial peripheral grooves, or flutes, cut radially inward to the central hollow bore at the proximal end of the male luer body 52. The fluted orifices 57 have radially projecting ribs 59 therebetween that structurally connect the end cap 58 to the luer body 52 and further assist in directing the fluid flow radially outward. The exemplary male luer shown in FIG. 11 comprises three radially symmetric fluted orifices. By increasing the number of radial flow paths, the overall plenum chamber has fewer and smaller zones of low flow compared to the metal insert luer example. As one of skill on the art will recognize, the orifices can be of any shape allowing flow of the fluid in a radial direction. For example, the orifices can have a rectangular shape. The number of orifices can be varied and the orifices can be symmetrically or asymmetrically spaced to enhance liquid mixing. For example, asymmetric spacing may impart different flow patterns that further improve cleaning efficiency. The ribs can also be shaped to improve mixing flow. The surfaces of the end cap can be shaped to further impart a change in the direction of flow.

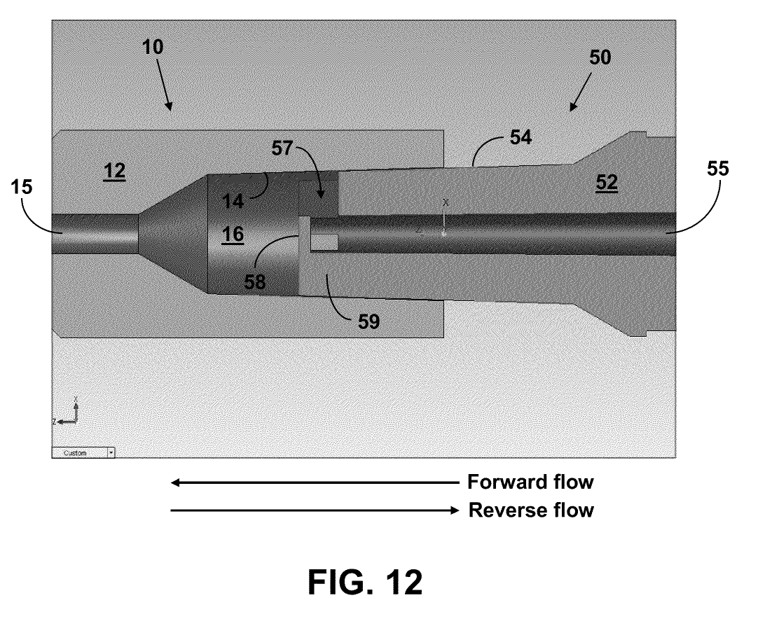

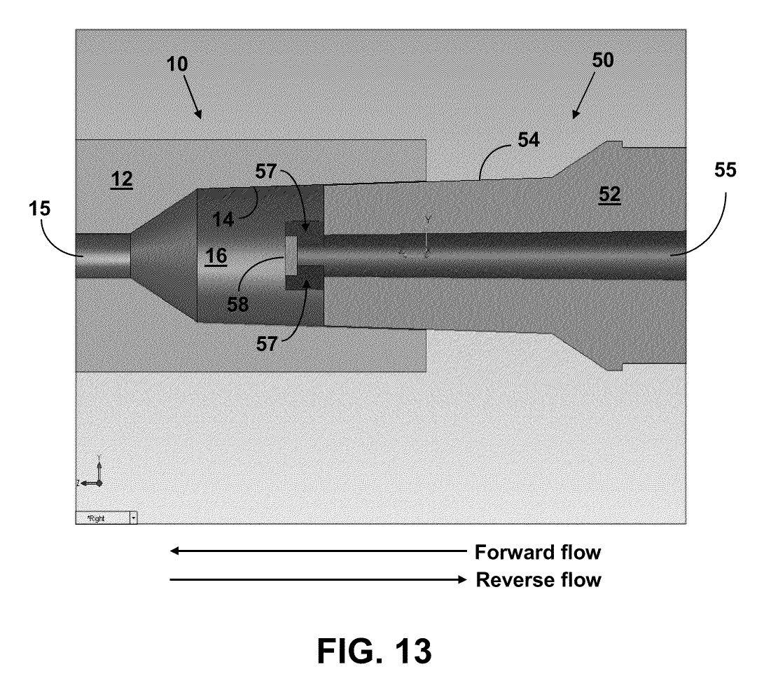

FIG. 12 shows a cross-section top-view schematic illustration, in the x-z plane, of a mixing luer connection comprising the mixing male luer 50 and a standard female luer 10. Radial flow is created as the fluid flows through the axial flow bore 55 and outwardly through the radial orifice 57, eventually striking the inner wall 14 of the plenum 16. FIG. 13 shows a cross-section side-view schematic illustration, in the y-z plane, of the same mixing luer connection, showing opposing radial orifices 57.

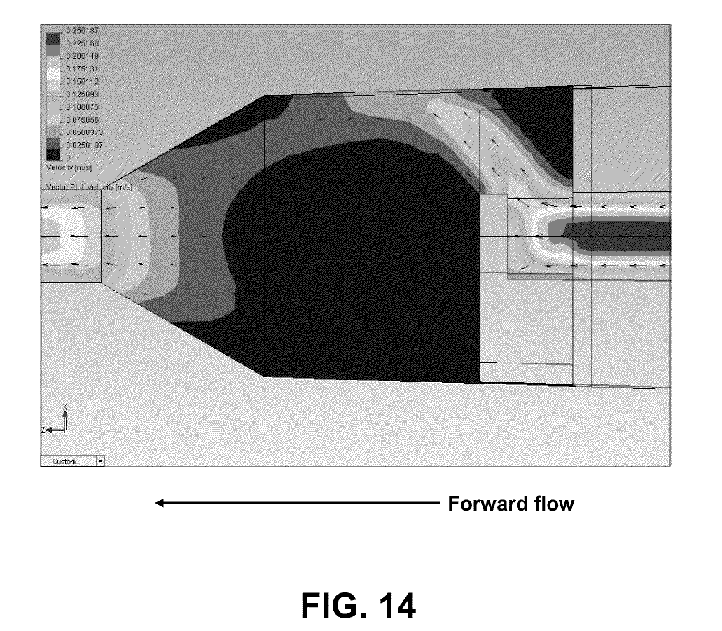

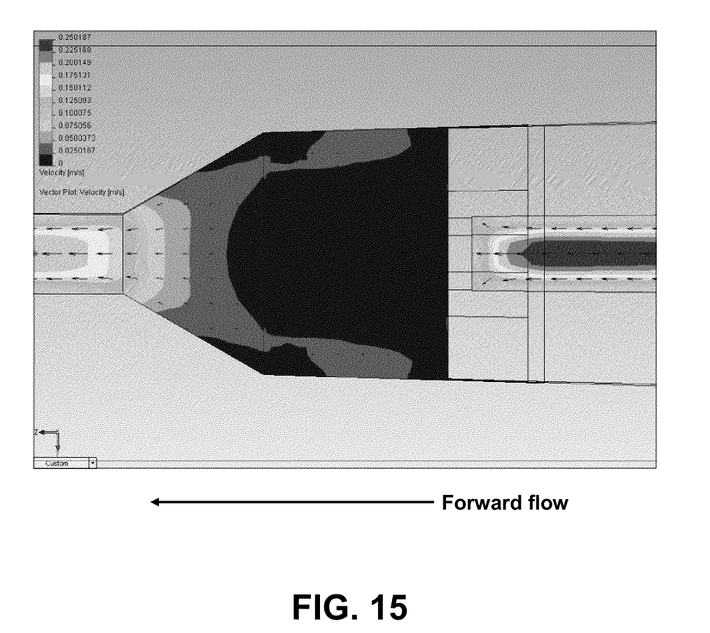

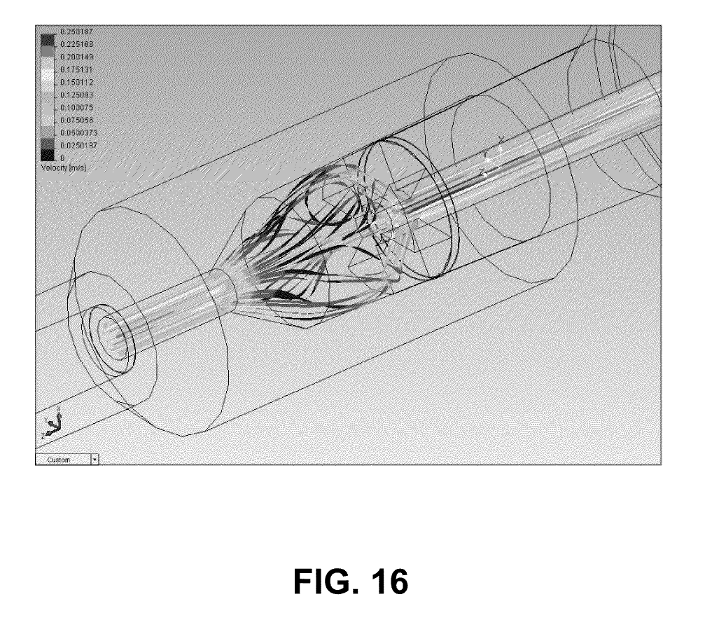

Fluid flow in the mixing luer connection was analyzed using a 3D CFD model. FIGS. 14-19 show the results of CFD analyses of the mixing luer connection. The mixing male luer, like the aforementioned tubular insert male luer, causes flow at the proximal end of the luer to be redirected radially outwards. However, unlike the tubular insert male luer, the mixing male luer causes the radial flow to impinge directly upon the inner walls of the female luer plenum. Further, the mixing luer can be entirely plastic. In forward flow, the axial jet stream becomes totally disrupted by the flat inner surface (wall) of the end cap that is perpendicular to the jet stream at the proximal end of the male luer flow passage bore, causing the flow to splay out radially through the orifices and into the plenum towards the inner walls of the female luer. This radial flow scours the plenum prior to converging inwards towards the female luer flow passage hole. FIG. 14 is a velocity contour plot at the center cross section of a mixing luer connection in the x-z plane with a 10 ml/min forward flow of water. Although there are dark areas denoting low-velocity zones, most are without recirculation. FIG. 15 is a velocity contour plot at the center cross section of the mixing luer connection in the y-z plane with a 10 ml/min forward flow of water. Again the dark areas represent low-velocity zones, but most are without recirculation. FIG. 16 is a 3D flow trajectory plot of a mixing luer connection showing 30 streamlines with a 10 ml/min forward flow of water. Examination of the streamlines shows that there are no large areas of stagnation present in the plenum chamber.

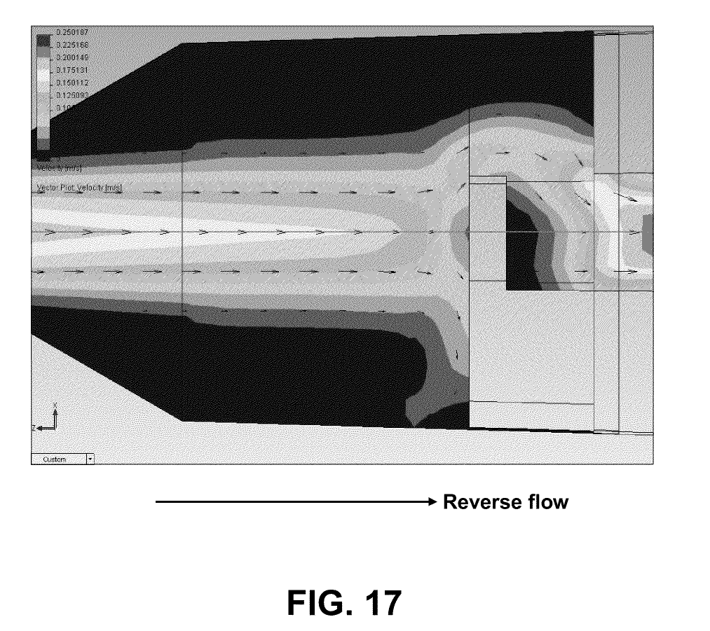

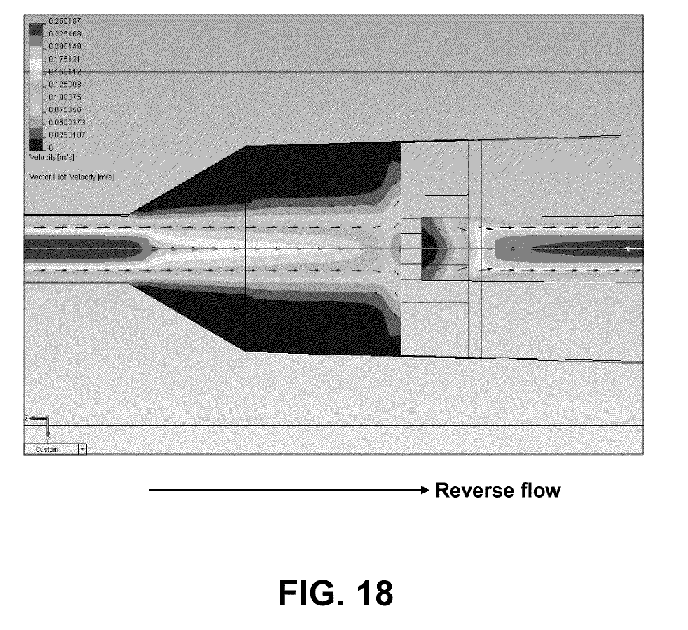

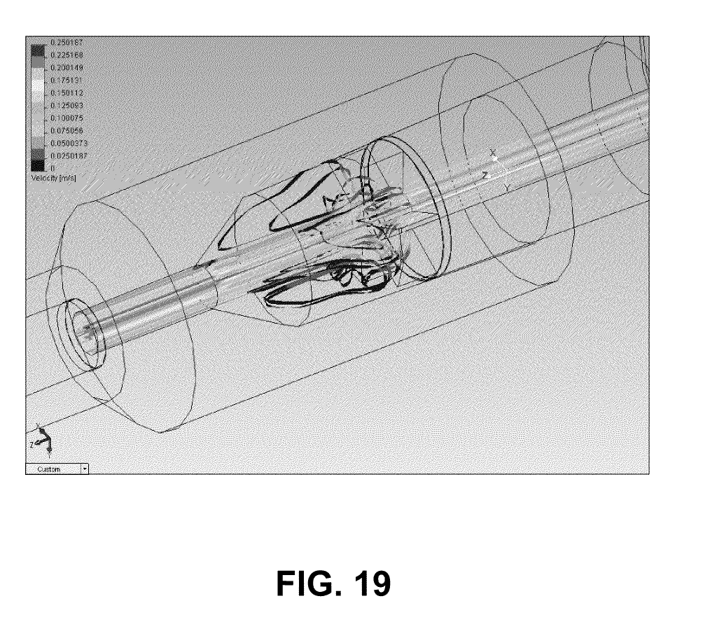

In reverse flow, the jet stream from the female luer side is likewise totally disrupted by the proximal-facing side of the male luer end cap, splaying flow radially outwards, then reversing inwards towards the orifices and into the male luer flow passage bore. FIG. 17 shows a velocity contour plot at center cross section of a mixing luer connection in the x-z plane with a 10 ml/min reverse flow of water. Dark areas are low-velocity zones, but mostly without recirculation. FIG. 18 is a velocity contour plot at center cross section of a mixing luer connection in the y-z plane with a 10 ml/min reverse flow of water. Dark areas are low-velocity zones, but mostly without recirculation. FIG. 19 is a 3D flow trajectory plot of a mixing luer connection showing 30 streamlines with a 10 ml/min reverse flow of water. As can be seen in these figures, some streamlines undergo a complete circulation loop before entering the orifices of the male luer, thus scouring the plenum.

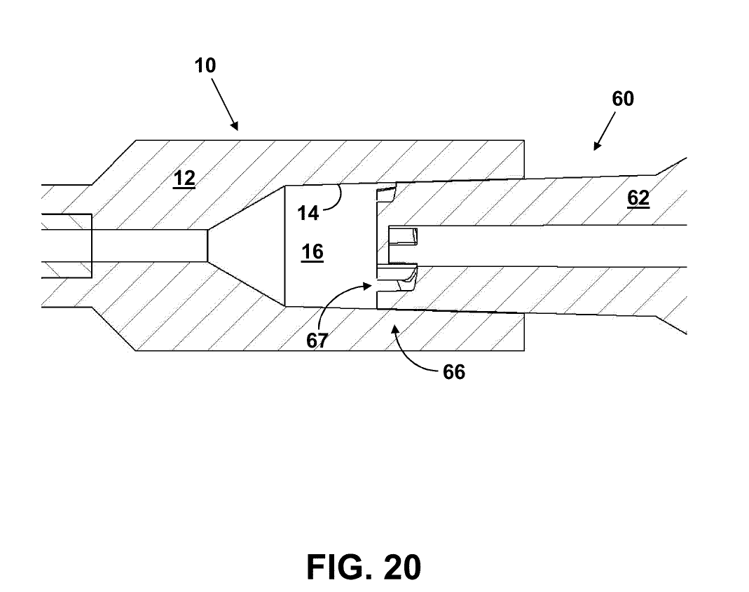

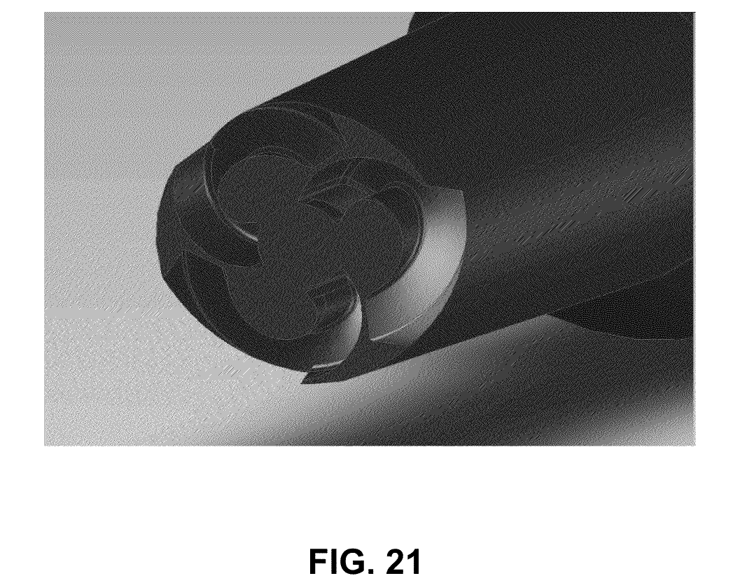

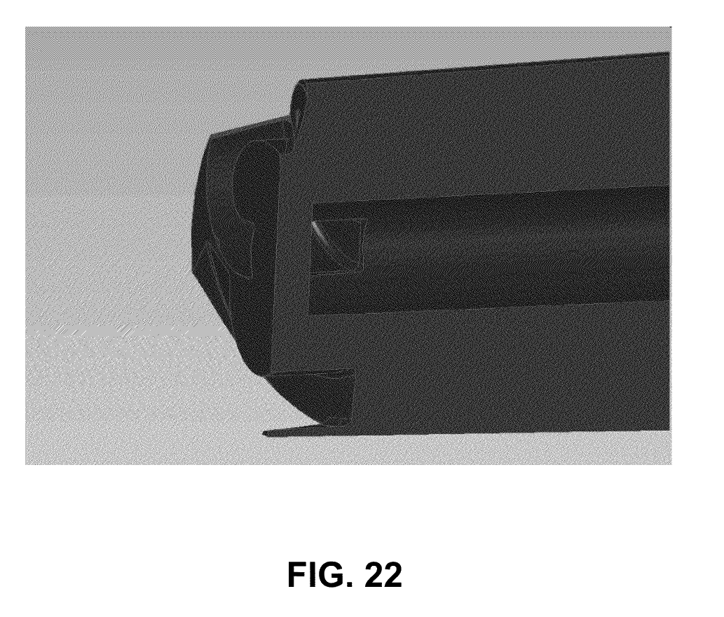

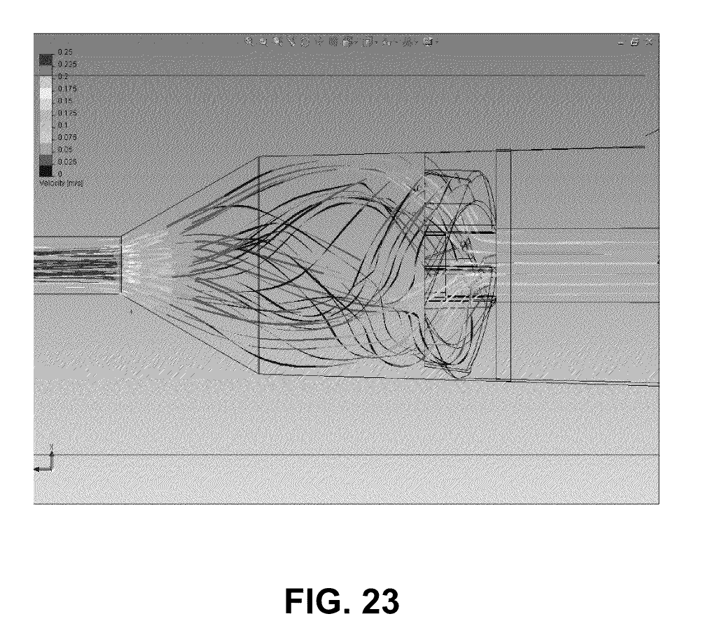

Based upon 3D CFD modeling, animal testing, and a general desire to reduce the overall shear stress on the blood at fixed flow rates, several additional luer types were developed that also promote liquid mixing. FIG. 20 shows a side-view schematic illustration of a “vortex” luer connection. FIGS. 21 and 22 show perspective end- and side-view illustrations of a vortex male luer 60. The vortex luer comprises a dispersing nozzle 66 having spiraling orifices 67 that impart a circumferential or rotational component to the fluid flow so that there are no appreciable areas of low flow in the plenum 16. The vortex luer also reduces the shear stress associated with blood striking the inner wall 14 of the female luer 10 at 90 degrees. FIG. 23 shows the streamlines of the vortex luer connection. Examination of this figure shows a swirling flow pattern that produces a thorough scrubbing of the plenum chamber with no significant areas of low flow.

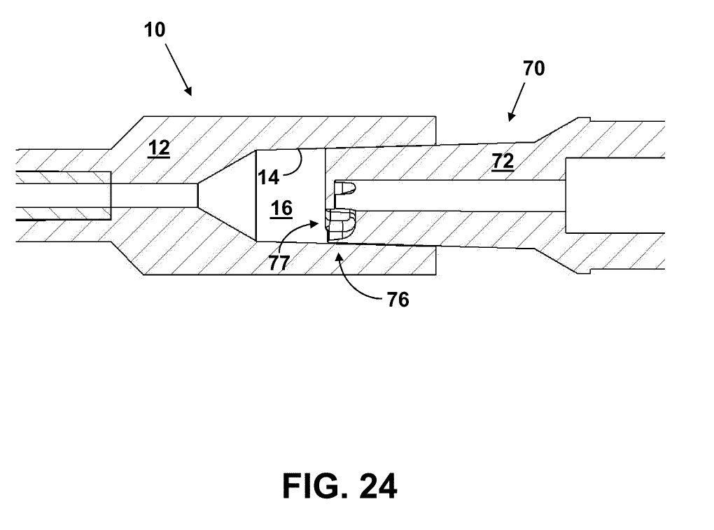

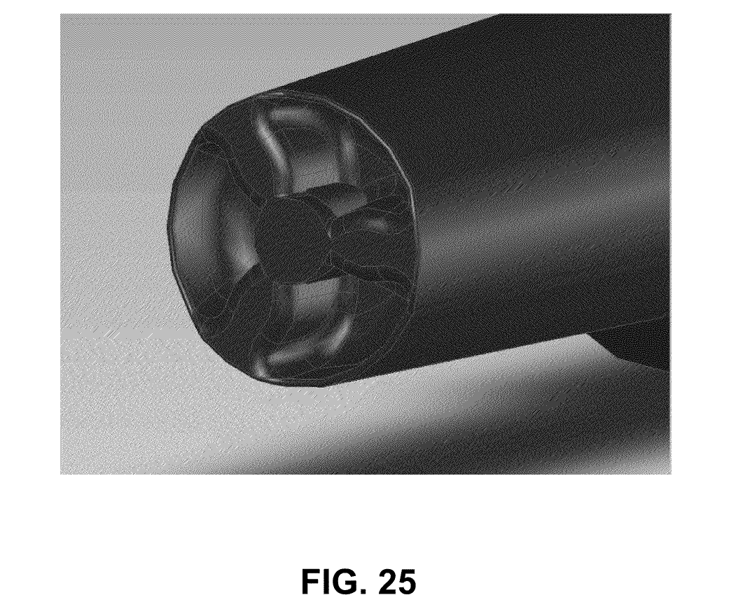

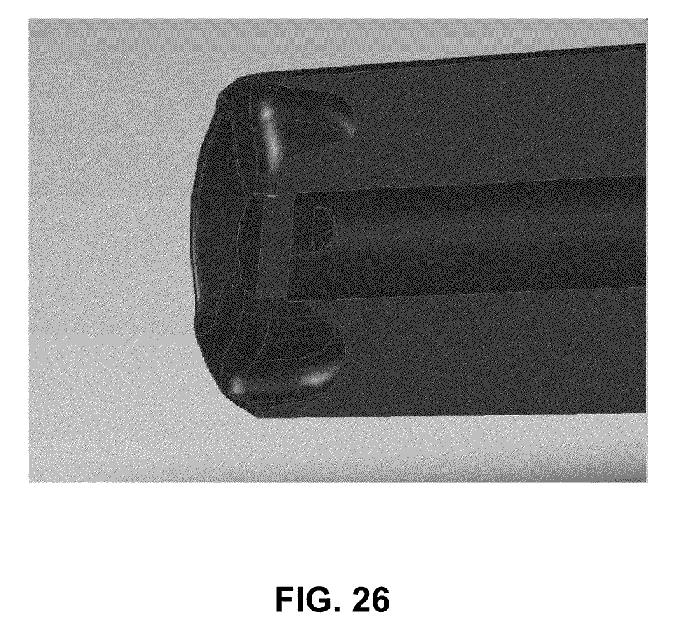

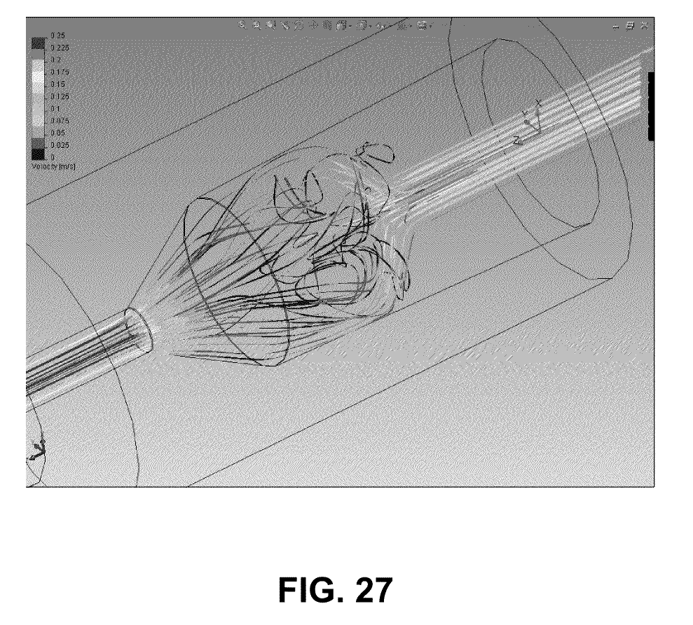

FIG. 24 shows a side-view schematic illustration of a “scoop” luer connection. FIGS. 25 and 26 show perspective end- and side-view illustrations of a scoop male luer. The scoop male luer 70 comprises a dispersing nozzle 76 having curved ribs that form scoop-shaped orifices 77 in the proximal end of the luer that produces a chaotic flow pattern such that the fluid scoops or “digs out” the plenum volume. FIG. 27 shows the streamlines of the scoop luer connection. Examination of this figure shows a more chaotic flow pattern but one that results in scrubbing of the plenum chamber and no significant areas of low flow. Further, a shear stress analysis indicated that the scoop luer created less shear then the mixing luer at comparable flow rates.

The scoop luer also changes its flow pattern as a function of flow rate to a degree greater than the other luer types. Specifically, the scoop luer has a flow pattern at a flow rate of 5 ml/min that is different than the flow pattern at 10 ml/min. The blood access system can be implemented so as to provide a push-stop or back-and-forth fluid movement pattern to maximize cleaning. The use of these fluid movements when coupled with a luer that changes flow patterns as a function of flow rate provides a luer connection that has exceptional cleaning effectiveness.

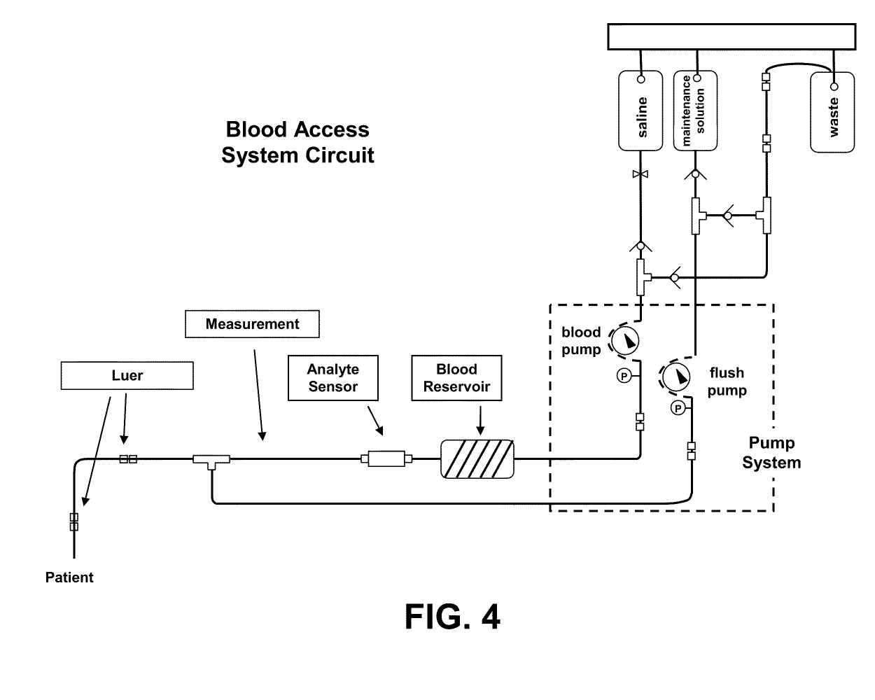

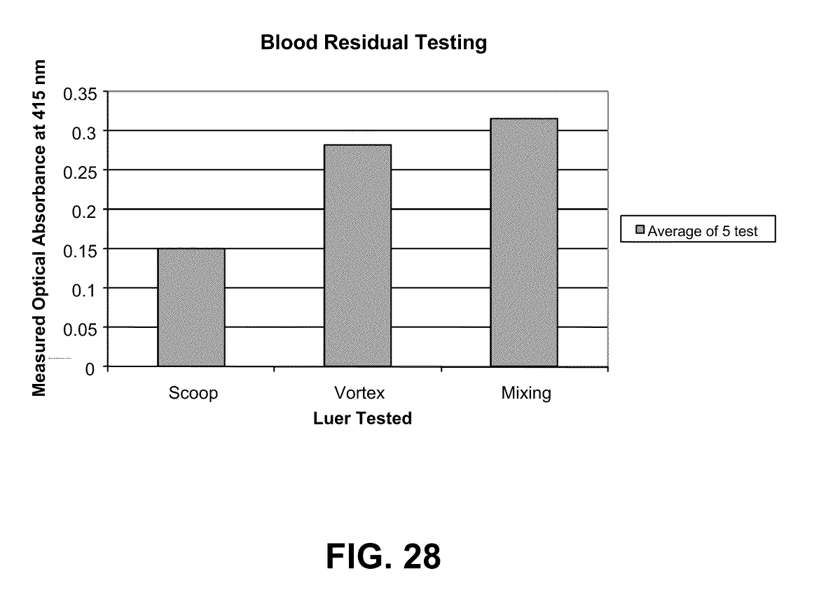

Tests were conducted to determine the cleaning effectiveness of the various luer types by measuring the residual matter remaining in a luer connection after a fixed cleaning cycle. The blood access system shown in FIG. 4 was used to conduct the luer tests. The system performs a blood withdrawal, followed by an infusion stage, and, finally, a cleaning process. To test the cleaning effectiveness of the various luer types, the total amount of saline used for the infusion and cleaning stages was reduced. Following ten draws of porcine blood adjusted to 50 Hct at 37° C., a vigorous cleaning of the luer connectors was conducted and the fluid was collected for analysis. The collected fluid was centrifuged to concentrate the residual cellular matter in the bottom of the tube. The matter was extracted to create fluid sample weighs of about 1.500 g. The optical absorbance of the resulting sample fluid was measured at 415 nm to determine the residual cellular matter. Five such tests were conducted for each luer type. The mixing, scoop, and vortex luer types were tested. A standard luer was not tested, since prior tests indicated the mixing luer had about 10× improved performance compared to the standard luer. FIG. 28 shows the results of the blood residual test comparisons. The scoop luer performed the best with the lowest average residual blood following the fixed cleaning protocol. The vortex luer was slightly better than the mixing luer. Subsequent animal tests based upon photographic evidence of the luer connections during testing on a non-heparinized porcine model confirmed the blood residual test comparisons.

The present invention has been described as medical luer connector that promotes improved cleaning characteristics. It will be understood that the above description is merely illustrative of the applications of the principles of the present invention, the scope of which is to be determined by the claims viewed in light of the specification. Exemplary luers described herein focus on changes to the male luer, since modifications to the female catheter placed in the patient were considered more problematic from a market perspective. However, similar changes to the female luer or both luers can result in improved cleaning effectiveness. These other variants and modifications of the invention will be apparent to those of skill in the art.

What is claimed is:

1. A male luer, comprising:
an elongated tubular male luer body comprising an axial hollow bore for the flow of fluid therethrough and having a proximal end that connects to a female luer thereby forming a plenum at the connection, and
a dispersing nozzle at the proximal of the male luer body comprising an axial end cap and one or more orifices in the sidewalls of the nozzle wherein fluid flowing axially in the hollow bore is redirected by the axial end cap to flow non-axially through the one or more orifices in the sidewalls of the nozzle into or out of the plenum.

2. The male luer of claim 1, wherein the one or more orifices impart a radial component to the redirected fluid flow.

3. The male luer of claim 2, wherein the one or more orifices comprise a radially projecting orifice.

4. The male luer of claim 1, wherein the one or more orifices impart a rotational component to the redirected fluid flow.

5. The male luer of claim 4, wherein the one or more orifices comprise a spiral orifice.

6. The male luer of claim 1, wherein the one or more orifices impart a chaotic flow pattern to the redirected fluid flow.

7. The male luer of claim 1, wherein the one or more orifices comprise a scooped orifice.

8. The male luer of claim 1, further comprising a tube inserted into the axial hollow bore of the male luer body and wherein the dispersing nozzle is at the proximal end of the inserted tube.

9. The male luer of claim 1, wherein the dispersing nozzle redirects the fluid flow along a first flow path in the plenum at a first rate of fluid flow and along a different flow path at a different rate of fluid flow.

10. The male luer of claim 1, wherein the dispersing nozzle comprises plastic or metal.

11. The male luer of claim 1, wherein the one or more orifices comprise radially symmetric orifices.

12. The male luer of claim 1, wherein the one or more orifices comprise radially asymmetric orifices.

13. The male luer of claim 1, wherein the one or more orifices comprise at least three orifices.
